(19) United States
(12) Patent Application Publication
(10) Pub. No.: US 2009/0138026 A1
(43) Pub. Date: May 28, 2009

(54) Water dermabrasion and skincare device and method thereof

(76) Inventor: Yueh-Chin Wu, Taipei (TW)

Correspondence Address: Meng-Tsuey Pan, P.O. Box 1-252, Sanchung, TW

(21) Appl. No.: 11/986,635

(22) Filed: Nov. 26, 2007

(51) Int. Cl. A61B 17/50

(52) U.S. Cl. 606/131

(57) ABSTRACT

A water dermabrasion and skincare device includes a water jet, a positive-pressure air compressor, a negative-pressure air compressor, a three-way flow control valve, a skincare container, a cuticle softening container and a waste water tank. The water jet is formed with a water outlet joint and a water inlet joint, which is connected to a water outputting end of the three-way flow control valve, while the water outlet joint is connected to the negative-pressure air compressor. Two water inputting ends of the three-way flow control valve are connected to a pure water bottle. When the three-way flow control valve is switched to a dermabrasion position or a skincare position, the negative-pressure air compressor can be started to generate a vacuum suction, which will suck pure water from the pure water bottle to flow into the water jet for executing a dermabrasion operation or a skincare operation.

### BACKGROUND OF THE INVENTION

1. Field of the Invention

The present invention relates to a device and a method for skincare and abrasive removal of the outer skin, and more particularly to a water dermabrasion and skincare device and a method thereof, wherein the single device can be optionally switched to execute a dermabrasion operation or a skincare operation, so as to simplify and speed up the dermabrasion and skincare operations.

2. Brief Discussion of the Related Art

A conventional dermabrasion instrument and a conventional water jet for dermatological treatment, similar to the present invention, are disclosed in U.S. Pat. No. 6,423,078 and No. 6,264,666 respectively. However, the dermabrasion instrument must simultaneously execute a flush operation and a single dermabrasion operation by an abrading member or an abrading end, resulting in lowering the speed, the convenience, and the efficiency of the operations. Furthermore, the water jet for dermatological treatment executes a dermabrasion operation and a skincare operation by simply jetting out pressurized water, wherein an abrasive substance must be added into the water to carry out the abrading effect, causing the operational inconvenience and limiting the entire efficiency.

### SUMMARY OF THE INVENTION

A primary objective of the present invention is to provide a water dermabrasion and skincare device and a method thereof; wherein one single device can be optionally switched to perform a dermabrasion or a skincare operation without adding any abrading substance into water, so as to simplify and speed up the dermabrasion and skincare operations.

A water dermabrasion and skincare device comprises a water jet (probe), a positive-pressure air compressor, a negative-pressure air compressor, a three-way flow control valve, a pure water bottle, a skincare container, a cuticle softening container and a waste water tank. A thick pen-like main body of the water jet has a rear end, which is connected with a water inlet joint and a water outlet joint. The water inlet joint is connected to a water outputting end of the three-way flow control valve via a water inputting pipe, while the water outlet joint is connected to the negative-pressure air compressor via a water outputting pipe. Furthermore, two water inputting ends of the three-way flow control valve are connected to the pure water bottle via two water pipes respectively. A second T-shaped pipe joint is installed on a water pipe for installing a first branch pipe to connect to the cuticle softening container. Moreover, a third T-shaped pipe joint is installed on another water pipe for installing a second branch pipe to connect to the skincare container. Since the skin debris and waste water generated during the dermabrasion operation, and the waste water generated during the skincare operation of the present invention should be collected and discarded, the waste water tank is provided at the water outputting pipe through which the water jet is connected to the negative-pressure air compressor for collecting the skin debris and the waste water.

In addition, a water pipe connected between the negative-pressure air compressor and the waste water tank is installed with a second adjusting valve and a negative-pressure gauge. A first T-shaped pipe joint is installed on the water inputting pipe for installing a third branch pipe to connect to the positive-pressure air compressor. The third branch pipe is further installed with an electromagnetic valve, a first adjusting valve and a positive-pressure gauge. Moreover, the water pipe connected between the second T-shaped pipe joint and the pure water bottle is installed with a third adjusting valve and a first check valve. The second branch pipe connected between the third T-shaped pipe joint and the skincare container is installed with a second flow regulator, while the first branch pipe connected between the second T-shaped pipe joint and the cuticle softening container is installed with a first flow regulator and a second check valve. The foregoing components comprise the water dermabrasion and skincare device of the present invention.

In the water jet (probe) of the present invention, the rear end of the main body is formed with the water inlet joint and the water outlet joint for being connected to the water inputting pipe and the water outputting pipe. In addition, the main body further has a front end provided with a head, a side wall provided with an electromagnetic valve switch, and a central portion provided with a water jetting pipe. The water jetting pipe has a rear end connected to the water inlet joint and the water inputting pipe, and a front end nearly close to a nozzle which is formed on a front terminal of the head. Furthermore, an inner portion of the main body adjacent to the water jetting pipe is further provided with a water returning pipe. The water returning pipe has a rear end connected to the water outlet joint and the water outputting pipe.

During execution of the dermabrasion operation of the water dermabrasion and skincare device, the handle of the three-way flow control valve is first switched to the dermabrasion position for turning on the power, so that the positive-pressure air compressor and the negative-pressure air compressor will be started to execute the dermabrasion operation. In the dermabrasion operation, the water jet can be held to make the nozzle of the head in direct contact with the skin layer to be abraded. The negative-pressure air compressor can generate the vacuum suction to suck and expand the pores on the skin layer. Meanwhile, the vacuum suction of the negative-pressure air compressor can guide the cuticle softening preparation in the cuticle softening container and the pure water in the pure water bottle to flow toward the water jet. Thus, when the electromagnetic valve switch on the side wall of the water jet is pushed down, the electromagnetic valve will be opened, so that the air pressure of the positive-pressure air compressor will pressurize a water flow in the water inputting pipe flowing toward the water jet. As a result, the water flowing into the water jetting pipe of the water jet will become a high-pressure spout which will be jetted toward the nozzle in contact with the skin layer, so as to be directly jetted to the skin layer at the dermabrasion site for generating a friction to carry out the effect of abrading and thinning the skin layer. After the dermabrasion operation, the waste water can be sucked into the waste water tank through the water returning pipe by the vacuum suction of the negative-pressure air compressor for storage and discard purposes. In the dermabrasion operation, the first adjusting valve, the second adjusting valve, and the first flow regulator can be adjusted, respectively, for controlling the flow of the pure water and cuticle softening preparation toward the water jet, and the jetting force of the water jet.

Furthermore, the water jet of the water dermabrasion and skincare device is held to execute the skincare operation, the handle of the three-way flow control valve is switched to the skincare position for only turning on the power to start the negative-pressure air compressor. As described above, the negative-pressure air compressor can generate the vacuum suction to suck and expand the pores on the skin layer to be cared. The fluid mixed with the skincare preparation in the skincare container and the pure water in the pure water bottle will simultaneously be sucked to flow into the water jetting pipe of the water jet, and then directly jetted to the pores on the skin layer where the nozzle of the head is in contact for executing the skincare operation. It is unnecessary to push down the electromagnetic valve switch to start the positive-pressure air compressor. After the skincare operation, the waste water can be sucked into the waste water tank through the water returning pipe by the vacuum suction of the negative-pressure air compressor for storage and discard purposes. In the skincare operation, the second adjusting valve and the second flow regulator can be adjusted, respectively, for controlling the flow of the pure water and the skincare preparation flowing toward the water jet.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a schematic deployment of a water dermabrasion and skincare device with water flowing directions during executing a dermabrasion operation according to a preferred embodiment of the present invention;

FIG. 2 is a schematic deployment of the water dermabrasion and skincare device with water flowing directions during executing a skincare operation according to the preferred embodiment of the present invention;

FIG. 3 is a side view of a water jet (probe) during executing the dermabrasion operation or the skincare operation according to the preferred embodiment of the present invention;

FIG. 4 is a side view of a flat-type head of the water jet (probe) with a smaller nozzle according to a preferred embodiment of the present invention;

FIG. 5 is a side view of a flat-type head of the water jet (probe) with a larger nozzle according to another preferred embodiment of the present invention; and

FIG. 6 is a side view of an oblique-type head of the water jet (probe) with a smaller nozzle according to further another preferred embodiment of the present invention.

### DETAILED DESCRIPTION OF THE PREFERRED EMBODIMENTS

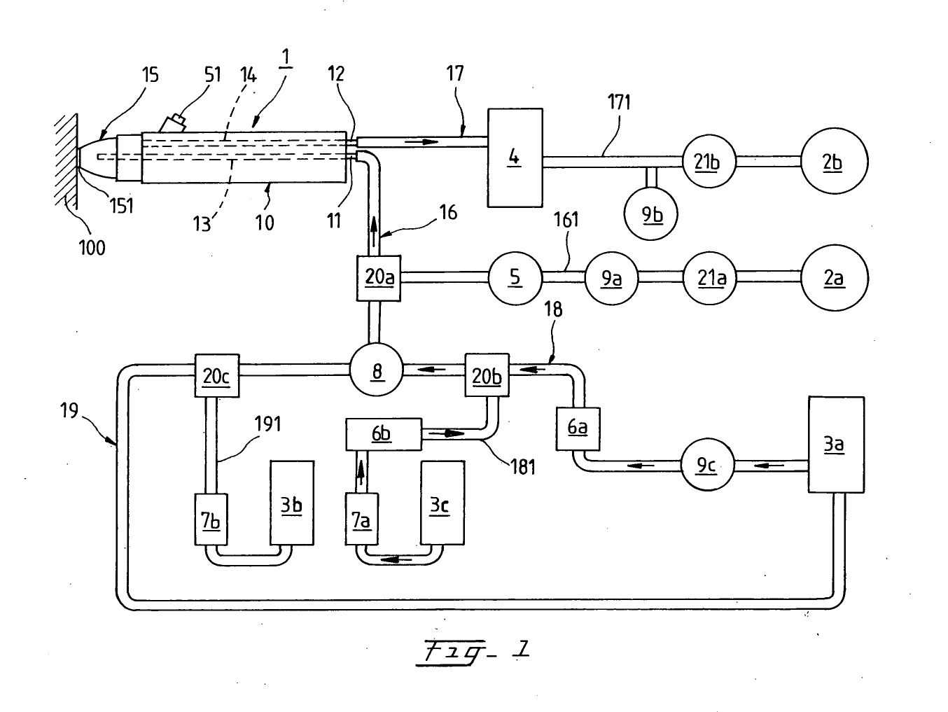

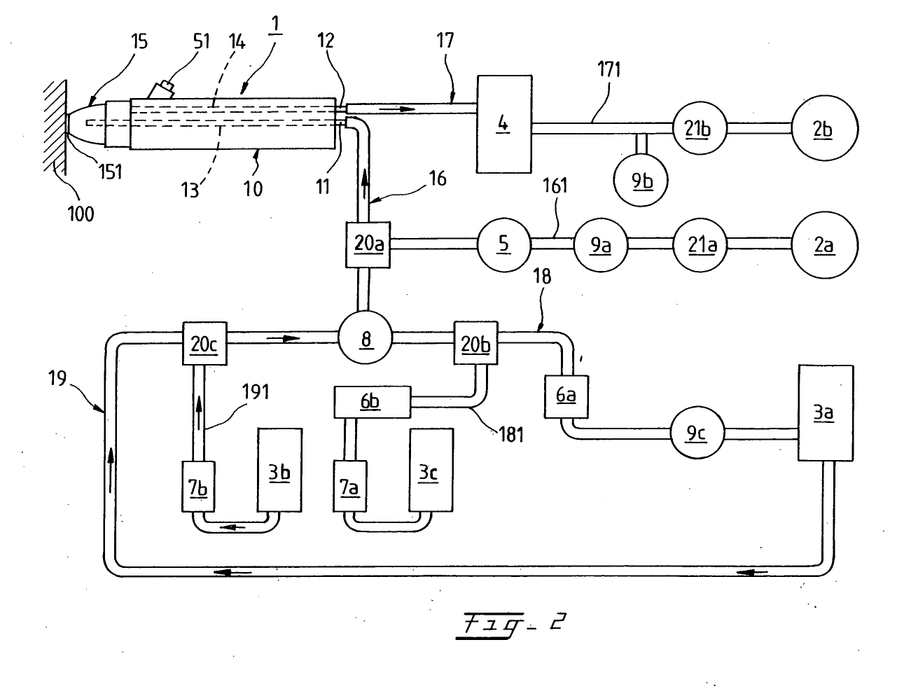

Referring to FIGS. 1 and 2, a water dermabrasion and skincare device according to a preferred embodiment of the present invention is illustrated. As shown, the water dermabrasion and skincare device comprises a water jet (probe) 1, a positive-pressure air compressor 2a, a negative-pressure air compressor 2b, a three-way flow control valve 8, a pure water bottle 3a, a skincare container 3b, a cuticle softening container 3c, and a waste water tank 4. A thick pen-like main body 10 of the water jet 1 has a rear end, which is formed with a water inlet joint 11 and a water outlet joint 12. The water inlet joint 11 is connected to a water outputting end of the three-way flow control valve 8 via a water inputting pipe 16, while the water outlet joint 12 is connected to the negative-pressure air compressor 2b via a water outputting pipe 17. Furthermore, two water inputting ends of the three-way flow control valve 8 are connected to the pure water bottle 3a via two water pipes 18, 19 respectively. When a handle (not shown) of the three-way flow control valve 8 is switched to a dermabrasion position or a skincare position, the negative-pressure air compressor 2b can be started to generate a vacuum suction which will suck pure water from the pure water bottle 3a, so that the pure water will flow to the water jet 1 for executing a dermabrasion operation or a skincare operation.

To satisfy needs of the dermabrasion operation and the skincare operation, a second T-shaped pipe joint 20b is installed on a water pipe 18 for installing a first branch pipe 181 to connect to the cuticle softening container 3c. Moreover, a third T-shaped pipe joint 20c is installed on another water pipe 19 for installing a second branch pipe 191 to connect to the skincare container 3b. Since the skin fragments and waste water generated during the dermabrasion operation of the present invention, and the waste water generated during the skincare operation of the present invention should be collected and discarded, the waste water tank 4 is provided at the water outputting pipe 17 through which the water jet 1 is connected to the negative-pressure air compressor 2b for collecting the skin fragments and the waste water.

In addition, a water pipe 171 connected between the negative-pressure air compressor 2b and the waste water tank 4 is installed with a second adjusting valve 9b and a negative-pressure gauge 21b. In the dermabrasion operation or the skincare operation, the second adjusting valve 9b can be adjusted to control the flow of the pure water running toward the water jet 1. Meanwhile, the negative-pressure gauge 21b can be visually checked to obtain a negative-pressure value of the negative-pressure air compressor 2b in operation. According to the water dermabrasion and skincare device of the present invention, except for installing the negative-pressure air compressor 2b, a first T-shaped pipe joint 20a is installed on the water inputting pipe 16 for installing a third branch pipe 161 to connect to the positive-pressure air compressor 2a. The third branch pipe 161 is further installed with an electromagnetic valve 5, a first adjusting valve 9a, and a positive-pressure gauge 21a. Thus, the electromagnetic valve 5 can be switched on or switched off to control if the positive-pressure value generated by the positive-pressure air compressor 2a pressurizes the flow rate of the pure water flowing toward the water jet 1 through the water inputting pipe 16. The first adjusting valve 9a can be adjusted to control an air pressure of the positive-pressure air compressor 2a. Meanwhile, the positive-pressure gauge 21a can be visually checked to obtain the air pressure value of the positive-pressure air compressor 2a in operation.

Moreover, the water pipe 18 connected between the second T-shaped pipe joint 20b and the pure water bottle 3a is installed with a third adjusting valve 9c and a first check valve 6a. The third adjusting valve 9c can be adjusted to control the flow running of the pure water toward the water jet 1 of the water dermabrasion and skincare device in the dermabrasion operation or the skincare operation. Meanwhile, the first check valve 6a can be used to control the pure water flowing toward the water jet 1, and prevent the pure water from flowing back to the pure water bottle 3a. The second branch pipe 191 connected between the third T-shaped pipe joint 20c and the skincare container 3b is installed with a second flow regulator 7b, while the first branch pipe 181 connected between the second T-shaped pipe joint 20b and the cuticle softening container 3c is installed with a first flow regulator 7a. The second flow regulator 7b is used to adjust the flow running of the water having a skincare preparation flowing toward the water jet 1 of the water dermabrasion and skincare device in the skincare operation, while the first flow regulator 7a is used to adjust the flow running of the water having a cuticle softening preparation flowing toward the water jet 1 during the dermabrasion operation. In the dermabrasion operation of the present invention, the positive-pressure air compressor 2a must be started. Thus, except for installing the first check valve 6a on the water pipe 18 connected to the pure water bottle 3a, the first branch pipe 181 connected to the cuticle softening container 3c must be installed with a second check valve 6b for preventing the cuticle softening preparation flowing toward the water jet 1 with the pure water from flowing back to the cuticle softening container 3c.

Referring to FIG. 3, in the water jet (probe) 1 of the present invention, the rear end of the main body 10 is formed with the water inlet joint 11 and the water outlet joint 12 for being connected to the water inputting pipe 16 and the water outputting pipe 17 respectively. In addition, the main body 10 further has a front end provided with a head 15, a side wall provided with an electromagnetic valve switch 51, and a central portion provided with a water jetting pipe 13. The water jetting pipe 13 has a rear end connected to the water inlet joint 11 and the water inputting pipe 16, and a front end nearly close to a nozzle 151 which is formed on a front terminal of the head 15. Furthermore, an inner portion of the main body 10 adjacent to the water jetting pipe 13 is further provided with a water returning pipe 14. The water returning pipe 14 has a rear end connected to the water outlet joint 12 and the water outputting pipe 17.

During execution of the dermabrasion operation of the water dermabrasion and skincare device, the handle (not shown) of the three-way flow control valve 8 is first switched to the dermabrasion position for turning on the power, so that the positive-pressure air compressor 2a and the negative-pressure air compressor 2b will be started to execute the dermabrasion operation. In the dermabrasion operation, the water jet (probe) 1 can be held to make the nozzle 151 of the head 15 in direct contact with a skin layer 100 to be abraded, as shown in FIG. 3. At this time, the negative-pressure air compressor 2b can generate the vacuum suction to suck and expand the pores on the skin layer to be abraded. Meanwhile, the vacuum suction of the negative-pressure air compressor 2b can guide the cuticle softening preparation in the cuticle softening container 3c and the pure water of the pure water bottle 3a to flow toward the water jet 1. Thus, when the electromagnetic valve switch 51 on the side wall of the water jet 1 is pressed, the electromagnetic valve 5 will be opened, so that the air pressure of the positive-pressure air compressor 2a will pressurize a water flow in the water inputting pipe 16 flowing toward the water jet 1. As a result, the water flowing into the water jetting pipe 13 of the water jet 1 will become a high-pressure spout which will be jetted toward the nozzle 151 in contact with the skin layer 100, so as to be directly jetted to the skin layer 100 at the dermabrasion site for generating a friction to carry out the effect of abrading and thinning the skin layer 100. After the dermabrasion operation, the waste water can be sucked into the waste water tank 4 through the water returning pipe 14 adjacent to the water jetting pipe 13 by the vacuum suction of the negative-pressure air compressor 2b for storage and discard purposes. In the dermabrasion operation, the first adjusting valve 9a, the second adjusting valve 9b, and the first flow regulator 7a can be adjusted, respectively, for controlling the flow of the pure water and the water mixed with the cuticle softening preparation toward the water jet 1, and the jetting force of the water jet 1.

Furthermore, the water jet 1 of the water dermabrasion and skincare device is held to execute the skincare operation, the handle (not shown) of the three-way flow control valve 8 is first switched to the skincare position for only turning on the power to start the negative-pressure air compressor 2b. As described above, the negative-pressure air compressor 2b can generate the vacuum suction to suck and expand the pores on the skin layer 100 to be cared. The water mixed with the skincare preparation in the skincare container 3b and the pure water in the pure water bottle 3a will simultaneously be sucked to flow into the water jetting pipe 13 of the water jet 1, and then directly jetted to the pores of the skin layer 100 where the nozzle 151 of the head 15 is in contact for executing the skincare operation. It is unnecessary to push down the electromagnetic valve switch 51 to start the positive-pressure air compressor 2a. After the skincare operation, the waste water can be sucked into the waste water tank 4 through the water returning pipe 14 adjacent to the water jetting pipe 13 by the vacuum suction of the negative-pressure air compressor 2b for storage and discard purposes. In the skincare operation, the second adjusting valve 9b and the second flow regulator 7b can be adjusted respectively, for controlling the flow of the pure water and the water mixed with the skincare preparation flowing toward the water jet 1.

The operations of the positive-pressure air compressor 2a and the negative-pressure air compressor 2b of the present invention need the related switches and power supplies, while the electromagnetic valve 5 and the electromagnetic valve switch 51 also need power supplies. Thus, it is necessary to install an integrated control device having a plurality of power supplies and switches for conveniently integrating the applied power supplies and optionally executing the dermabrasion operation or the skincare operation.

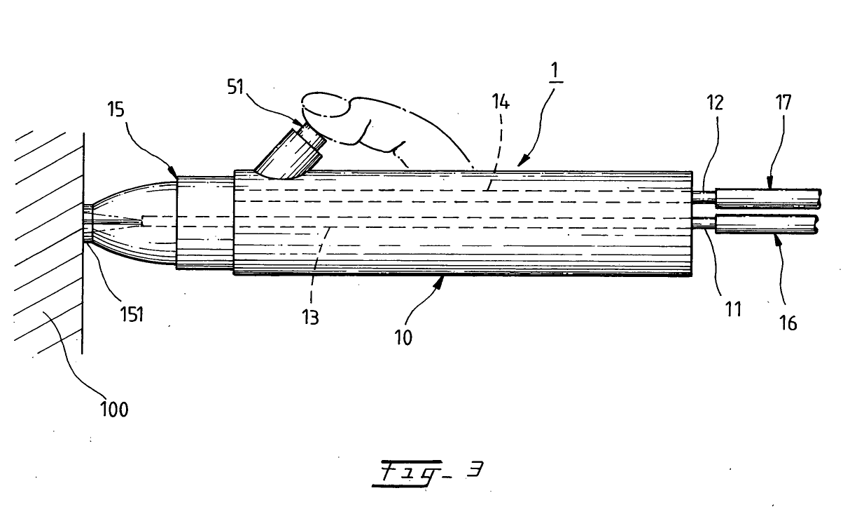

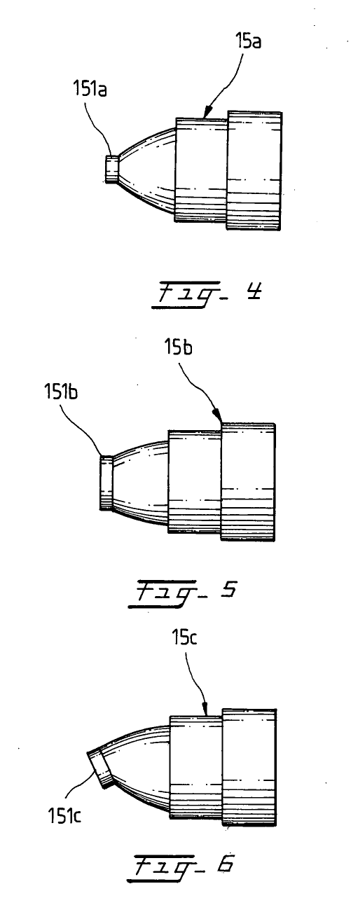

Referring to FIGS. 4, 5 and 6, because a skin layer at the dermabrasion site or a skincare layer may have various areas, while the skin layer may be a planar surface, a vertical surface, or an oblique surface, the head 15 of the water jet 1 of the water dermabrasion and skincare device of the present invention may be selected from various heads of various specifications, such as a head 15a having a flat nozzle 151a with a smaller diameter as shown in FIG. 4, a head 15b having a flat nozzle 151b with a larger diameter as shown in FIG. 5, or a head 15c having an oblique nozzle 151c with another diameter as shown in FIG. 6.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A water dermabrasion and skincare device comprising a water jet, a positive-pressure air compressor, a negative-pressure air compressor, a three-way flow control valve, a pure water bottle, a skincare container, a cuticle softening container and a waste water tank;
said water jet being a thick pen-like main body, including:
a water inlet joint and a water outlet joint formed on a rear end of the main body, respectively;
a head having a front terminal formed with a nozzle and formed on a front end of the main body;
a water jetting pipe formed in a central portion of the main body, said water jetting pipe having a rear end connected to said water inlet joint and a front end nearly close to said nozzle;
a water returning pipe formed in an inner portion of the main body and adjacent to said water jetting pipe;
an electromagnetic valve switch formed on a side wall of the main body;
said three-way flow control valve having a water outputting end installed with a water inputting pipe for being connected to said water inlet joint on the rear end of said water jet;
said negative-pressure air compressor installed with a water outputting pipe for being connected to said water outlet joint on the rear end of said water jet;
said pure water bottle receiving pure water and being connected to two water inputting ends of the three-way flow control valve via two water pipes, respectively;
said skincare container receiving pure water or liquid mixed with a skincare preparation and installed with a second branch pipe for being connected to a third T-shaped pipe joint through which one of the two water pipes of the pure water bottle is connected to one of the two water inputting ends of said three-way flow control valve;
said cuticle softening container receiving pure water or liquid mixed with a cuticle softening preparation and installed with a first branch pipe for being connected to a second T-shaped pipe joint through which the other of the two water pipes of the pure water bottle is connected to the other of the two water inputting ends of said three-way flow control valve;
said waste water tank installed on said water outputting pipe connected to said negative-pressure air compressor, wherein a water pipe connected between said negative-pressure air compressor and said waste water tank is installed with a second adjusting valve and a negative-pressure gauge;
said positive-pressure air compressor installed with a third branch pipe for being connected to a first T-shaped pipe joint installed on said water inputting pipe, said third branch pipe installed with an electromagnetic valve, a first adjusting valve and a positive-pressure gauge;
said pure water bottle connected with two water pipes, having disposed:
a third adjusting valve and a first check valve installed on one of the water pipes connected between said second T-shaped pipe joint and said pure water bottle;
a second check valve and a first flow regulator installed on said first branch pipe connected between said second T-shaped pipe joint and said cuticle softening container; and
a second flow regulator installed on said second branch pipe connected between said third T-shaped pipe joint and said skincare container.

2. The water dermabrasion and skincare device of claim 1, wherein the head of the water jet is a head having a flat or oblique nozzle with various diameters.

3. The water dermabrasion and skincare device of claim 1, wherein the nozzle of the head of the water jet is in direct contact with a skin layer at a dermabrasion site when the water jet is in the dermabrasion operation, so that the negative-pressure air compressor generates a vacuum suction to suck and expand pores on the skin layer at the dermabrasion site, while the vacuum suction of the negative-pressure air compressor guides a liquid in the cuticle softening container and the pure water in the pure water bottle to flow toward the water jet, so that the electromagnetic valve switch on the side wall of the water jet is pushed down to have the electromagnetic valve opened, and an air pressure of the positive-pressure air compressor pressurizes a water flow in the water inputting pipe flowing toward the water jet, and the water flowing into the water jetting pipe of the water jet can become a high-pressure spout which can be jetted toward the nozzle in contact with the skin layer, so as to be directly jetted to the skin layer for generating a friction to carry out effects of abrading and thinning the skin; and wherein only the vacuum suction of the negative-pressure air compressor sucks and expands pores on the skin layer to be cared when the water jet is in the skincare operation, so that a liquid in the skincare container and the pure water in the pure water bottle are simultaneously sucked to flow into the water jetting pipe of the water jet, and then directly jetted to the pores on the skin layer where the nozzle of the head is in contact for executing the skincare operation.
